(19) United States
(12) Patent Application Publication
(10) Pub. No.: US 2022/0226410 A1
(43) Pub. Date: Jul. 21, 2022

(54) CACTI AND CACTI PARTS FOR COSMETIC TREATMENT OF SKIN AND BALDNESS AND HAIR REGENERATION AND REGROWTH

(71) Applicant: Isaak HAYIK, Yehud (IL)

(72) Inventor: Isaak HAYIK, Yehud (IL)

(21) Appl. No.: 17/577,935

(22) Filed: Jan. 18, 2022

Related U.S. Application Data
(60) Provisional application No. 63/138,535, filed on Jan. 18, 2021.

(51) Int. Cl. A61K 36/33; A61K 9/00

(52) U.S. Cl. CPC A61K 36/33; A61K 9/0014

(57) ABSTRACT

The invention pertains to cosmetic preparations based on the biome of cacti plants, namely the body of cacti plants, cacti plants fruits and derivatives of cacti plants. Such preparations are suitable for topical or dermatological treatment of skin, scalp skin, hair loss, baldness and hair regeneration. The active ingredient of such composition or preparation for such cosmetic topical application or personal care treatment includes selected relative amounts of extracts and parts of Opuntia ficus-indica of finely ground fruit or fruit extract; finely ground fruit skin or fruit skin extract; finely ground cladodes; finely ground areoles; finely ground spines and finely ground glochids of Opuntia ficus-indica. A suitable medium is used in the preparation or composition for particular applications for skin, skin scalp and near environment of hair follicles. The composition is suitable for cosmetic or dermatological treatment of skin, scalp skin, hair loss, baldness and hair regeneration.

### TECHNICAL FIELD

The present invention pertains to cosmetic preparations that are based on the biome of cacti plants, namely the body of cacti plants, cacti plants fruits, derivatives of cacti plants. More particularly, the present invention pertains to such preparations for topical or dermatological treatment of skin, scalp skin, hair loss, baldness and hair regeneration.

### BACKGROUND

Cosmetic preparations, which are based on natural ingredients, are well-known in the art. Different species of plants contain nutrients that are useful for human health and compounds with healing and rejuvenating powers to the human biology. For this reason, such plants are used in almost their natural form for treating cosmetic or medical conditions. Cacti plants are considered a rich source of such nutrients and compounds and have been extensively investigated in this respect. Substantial attempts have been made so far to manufacture different pharmaceutical and cosmetic preparations and compositions that contain the unique combination of nutrients and compounds in cacti plants. See for example, U.S. Pat. Nos. 5,000,949 and 6,210,738.

The prior art also describes a variety of compositions and preparations for grooming hair, treating skin of the scalp, hair loss to repair, replenish and enrich the epidermal environment of hair follicles with effective nutrients to rejuvenate hair growth, overcome negative effects of inhibitors in such environment and overcome disorders, which are related to loss of hair, e.g. alopecia areata. DE 4 331 252 describes a liquid composition for oral administration that contains pressed juice of merismatic tissue of tropical green leaf plants such as cactus types and pampas grass. A hair gel for external application is also disclosed therein.

It is, therefore, an object of the present invention to provide compositions and preparations based on extracts of cacti plants for cosmetic and dermatological uses and in different types of chemical vehicles particularly for topical treatment of the skin, scalp skin, hair loss, baldness and hair regeneration.

### SUMMARY

In one aspect, the present invention pertains to a composition or preparation for cosmetic topical application or personal care treatment that comprises extract of parts of Opuntia ficus-indica comprising fresh skins of fruits and grounded spiny stalks and leaves of said Opuntia ficus-indica;

a suitable medium for the specific type of composition or preparation and their particular application to the skin, skin scalp and near environment of hair follicles;

and

preservatives and additives.

The following table summarizes the various materials and amounts per 100 gr found in fruits of Opuntia ficus-indica:

TABLE I

| Nutrient | Amount (per 100 gr) | |
|---|---|---|
| Calories | 41 | Kcal |
| Proteins | 0.73 | g |
| Carbohydrates | 9.57 | g |
| Fats | 0.51 | g |
| Saturated Fat | 0.067 | g |
| Cholesterol | 0 | |
| Sodium | 5 | mg |
| Fibers | 3.6 | g |
| Water | 87.55 | g |
| Vitamin A | 2 | μg |
| Vitamin B (total) | 0.6 | mg |
| Vitamin B1 | 0.014 | mg |
| Vitamin B2 | 0.06 | mg |
| Vitamin B3 - Niacin | 0.46 | mg |
| Vitamin B6 | 0.06 | mg |
| Folic acid (Vitamin B9) | 6 | μg |
| Vitamin C | 14 | mg |
| Calcium | 56 | mg |
| Iron | 0.3 | mg |
| Magnesium | 85 | mg |
| Phosphorus | 24 | mg |
| Zinc | 0.12 | mg |
| Potassium | 220 | mg |
| Fatty acids (multi- non-saturated) | 0.213 | g |
| α-Linolenic acid (Omega 3) | 0.023 | g |
| Linoleic acid (Omega 6) | 0.186 | g |
| Fatty acids (non-saturated) | 0.075 | g |
| Oleic acid (Omega 9) | 0.072 | g |

The table above exemplifies the potency of the Opuntia ficus-indica plant and hence their potential as efficient material that has therapeutic effect. Particularly, the invention comprises a pharmaceutical composition that comprises the following parts and/or extracts of Opuntia ficus-indica: a selected amount of finely ground fruit or fruit extract; a selected amount of finely ground fruit skins or fruit skin extract; a selected amount of finely ground cladodes; a selected amount of finely ground areoles; a selected amount of finely ground spines and a selected amount of finely ground glochids of Opuntia ficus-indica, where such composition has antiviral, anti-inflammatory, antioxidant effects in a patient in need of treatment of disorders and diseases related to such effects.

In another aspect, the present invention pertains to a composition or preparation for cosmetic topical application or personal care treatment that comprises extract of parts of Opuntia ficus-indica comprising: a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;

a suitable medium for the specific type of composition or preparation and their particular application to the skin, skin scalp and near environment of hair follicles;

and

preservatives and additives,

wherein said composition is suitable for cosmetic or dermatological treatment of skin, scalp skin, hair loss, baldness and hair regeneration.

In still another aspect, the present invention pertains to a composition or preparation for cosmetic topical application or personal care treatment that comprises extract of parts of Opuntia ficus-indica comprising: a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;

a suitable medium for the specific type of composition or preparation and their particular application to the skin, skin scalp and near environment of hair follicles;

and

preservatives and additives,

wherein the composition or preparation is provided in one or more of the following forms: cream, lotion, tincture, oil, spray, spray mist, soaked pads, powder pads, powder, serum, shampoo and soap.

In one embodiment, the composition or preparation of the present invention comprises additives such as fragrances and perfumes.

In another embodiment, the composition or preparation of the present invention comprises additives such strengthening active substances in addition to the active ingredients in the cacti extract.

In still another embodiment, the composition or preparation of the present invention is oil-based or aqueous-based.

In still another embodiment, the composition or preparation of the present invention is applied on dried skin.

In still another embodiment, the composition or preparation of the present invention is applied on moist skin.

In still another embodiment, the composition or preparation of the present invention is applied on skin which is sanitized with an alcoholic agent.

In still another embodiment, the composition or preparation of the present invention is absorbable inside the external layers of the skin.

In still another embodiment, the composition or preparation of the present invention is removable off of the skin with suitable oil-based or aqueous-based agent.

In one aspect, the composition or preparation of the present invention is applied in a particular regimen for long term results. In particular, such composition or preparation may be applied on a daily regimen, twice a day, at specific hours of the day, every other day, in selected days of the week and in general in selected time intervals between applications for optimal results and avoidance of accumulated side- or negative effects.

In another aspect, the composition or preparation of the present invention is applied for reconstruction or rehabilitation of damaged skin top tissue after medical treatment, surface wound healing or injury.

Table II below details the relative amounts of the active ingredients taken form Opuntia ficus-indica and used in pharmaceutical compositions for treating antiviral, antibacterial and/or antioxidant related disorders and diseases:

TABLE II

| | Range of relative amount of active |
|---|---|
| | ingredients in the pharmaceutical |
| Ingredients of Opuntia ficus-indica | composition (% w/w) |
| Finely ground fruit | 0.5-50 |
| Fruit extract | 0.55-15 |
| Fruit skins | 0.5-25 |
| Fruit skin extract | 0.5-15 |
| Finely ground cladodes | 0.5-25 |
| Finely ground areoles | 0.5-30 |
| Finely ground spines | 0.5-30 |
| Finely ground glochids | 0.5-30 |

Table III below details the relative amounts of ingredients of the pharmaceutical composition that comprises active ingredients taken from Opuntia ficus-indica for treating antiviral, antibacterial and/or antioxidant related disorders and diseases:

TABLE III

| | Range of relative amount of |
|---|---|
| Ingredient of pharmaceutical | ingredients in the pharmaceutical |
| composition comp Opuntia ficus-indica | composition (% w/w) |
| Active ingredient based on parts and | 5-15 |
| extracts of Opuntia ficus-indica | |
| an alcoholic, lipid, oily or hydrous | 40-75 |
| medium | |
| preservatives and additives | 5-10 |

Table IV below details the relative amounts of the ingredients of a pad that comprises a pharmaceutical composition that comprise active ingredients taken from Opuntia ficus-indica for treating antiviral, antibacterial and/or antioxidant related disorders and diseases:

TABLE IV

| | Range of relative amount of |
|---|---|
| Ingredient of pharmaceutical | ingredients in the pharmaceutical |
| composition comp Opuntia ficus-indica | composition (% w/w) |
| Active ingredient based on parts and | 5-15 |
| extracts of Opuntia ficus-indica | |
| a polymeric matrix; | 40-75 |
| preservatives and additives | 5-10 |

1. A composition or preparation for cosmetic topical application or personal care treatment comprising selected relative amounts of extracts and parts of Opuntia ficus-indica comprising: a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;
a suitable medium for the specific type of composition or preparation and their particular application to skin, skin scalp and near environment of hair follicles;
and
preservatives and additives,
wherein said composition is suitable for cosmetic or dermatological treatment of skin, scalp skin, hair loss, baldness and hair regeneration.

2. The composition or preparation for cosmetic topical application or personal care treatment according to claim 1, wherein said composition or preparation is provided in a form selected from cream, lotion, tincture, oil, spray, spray mist, soaked pads, powder pads, powder, serum, shampoo and soap.

3. The composition or preparation according to claim 1, further comprising additives selected from fragrances and perfumes.

4. The composition or preparation according to claim 1, further comprising additives selected from strengthening active substances in addition to active ingredients of said Opuntia ficus-indica.

5. The composition or preparation according to claim 1, wherein said composition or preparation is oil-based.

6. The composition or preparation according to claim 1, wherein said composition or preparation is aqueous-based.

7. The composition or preparation according to claim 1, wherein said composition or preparation is applicable on dried skin.

8. The composition or preparation according to claim 1, wherein said composition or preparation is applicable on moist skin.

9. The composition or preparation according to claim 1, wherein said composition or preparation is applicable on skin which is sanitized with an alcoholic agent.

10. The composition or preparation according to claim 1, wherein said composition or preparation is absorbable inside the external layers of the skin.

11. The composition or preparation according to claim 1, wherein said composition or preparation is removable off of a skin with suitable oil-based or aqueous-based agent.

12. The composition or preparation according to claim 1, wherein said composition or preparation is applicable in a particular regimen for long term results.

13. The composition or preparation according to claim 1, wherein said regimen is selected from daily regimen, twice a day, at specific hours of a day, every other day, in selected days of a week and in selected time intervals between applications for optimal results and avoidance of accumulated side- or negative effects.

14. The composition or preparation according to claim 1, wherein said composition or preparation is applicable for reconstruction or rehabilitation of damaged skin top tissue after medical treatment, surface wound healing or injury.

15. The composition or preparation according to claim 1, wherein said selected relative amounts of said extracts and parts of Opuntia ficus-indica are 0.5-50% w/w of finely ground fruit and/or 0.5-15% w/w of fruit extract; 0.5-25% w/w of fruit; 0.5-25% w/w of finely ground cladodes; 0.5-30% w/w of of finely ground areoles; 0.5-30% w/w of finely ground spines and 0.5-30% w/w of finely ground glochids.

16. The composition or preparation according to claim 1, wherein relative amounts of components of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.

17. The composition or preparation according to claim 1, wherein said composition is provided in a pad, and wherein relative amounts of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.
